(19) United States
(12) Patent Application Publication
(10) Pub. No.: US 2020/0390613 A1
(43) Pub. Date: Dec. 17, 2020

(54) BANDAGE DEVICE AND METHODS OF USE AND MANUFACTURE THEREOF

(71) Applicants: Nanette R. Mendez, San Juan, PR (US); Sandy M. Gonzalez, Santurce, PR (US)

(72) Inventors: Nanette R. Mendez, San Juan, PR (US); Sandy M. Gonzalez, Santurce, PR (US)

(21) Appl. No.: 16/441,875

(22) Filed: Jun. 14, 2019

(51) Int. Cl. A61F 13/14; A61F 13/02

(52) U.S. Cl. CPC A61F 13/145; A61F 2013/15016; A61F 13/0236; A61F 13/0203

(57) ABSTRACT

A device comprising: a breast bandage hosting a cup portion, a reservoir portion, a gauze portion, and an adhesive portion, wherein the cup portion hosts the reservoir portion, wherein the gauze portion extends about the reservoir portion, wherein the adhesive portion is external to the reservoir portion.

### TECHNICAL FIELD

Generally, this disclosure relates to bandage devices. More particularly, this disclosure relates to post-operative bandages for use after surgeries, procedures and injuries.

### BACKGROUND

After a surgery (e.g., cosmetic, reconstructive, mastectomy) at an external surgery site (e.g., chest, breast) of a patient, the surgery site or its proximate areas (e.g., lateral, stomach) may have various drainage tubes extending therefrom in order to remove various bodily liquids (e.g., blood, lymph) for expedited healing of the patient. Also, properly bandaging a patient often takes time and expertise, which many caregivers do not have. Due to the surgery, the patient may be experiencing pain, numbness, swelling, or tingling in the surgery site, or its proximate areas. When the surgery, for example, is breast surgery, the pain may even extend to the shoulders, or upper arms. As such, the patient can be dressed with a specialized bra over the surgery site, which may also cover the proximate areas. However, the specialized bra may be interfering with the drainage tubes, overly compressing or rough. Other bandages may also be interfering with the drainage tubes, overly compressing or rough, unable to accommodate swelling, difficult to self-dress, lack an infection detection sensor or an anti-bacterial, anti-inflammation, or medicinal composition.

### SUMMARY

In an embodiment, a device comprises: a bandage, such as for example, a bandage for the breast, chest, stomach, armpit, lateral side, shoulder, arm, elbow, wrist, hand, finger, hip, buttocks, neck, torso, thigh, knee, calf, ankle, heel, foot, toe, head, chin, nose, jaw, or any other body location, with the bandage hosting a cup portion, a reservoir portion, a gauze portion, and an adhesive portion, wherein the cup portion hosts the reservoir portion, wherein the gauze portion extends about the reservoir portion, wherein the adhesive portion is external to the reservoir portion.

### DESCRIPTION OF DRAWINGS

These figures illustrate various embodiments of this disclosure. Such figures are not to be construed as necessarily limiting of this disclosure. Like numbers or similar numbering scheme can refer to like or similar elements throughout.

FIG. 1 illustrates a rear view of an embodiment of a bandage according to this disclosure.

FIG. 2 illustrates a lateral view of an embodiment of a bandage according to this disclosure.

FIGS. 3a-3c illustrate various views of an embodiment of a bandage on a torso of a mannequin or a human according to this disclosure.

FIG. 4 illustrates a rear view of an embodiment of a bandage according to this disclosure.

FIG. 5 illustrates a rear view of an embodiment of a bandage according to this disclosure.

FIG. 6 illustrates a rear view of an embodiment of a bandage according to this disclosure.

FIGS. 7a-7b illustrate an embodiment of usage of a drain port according to this disclosure.

### DETAILED DESCRIPTION

Generally, this disclosure enables an integrated bandage system to serve as a post-operative bandage for placement or positioning over or in effective proximity of a wound area (e.g., breast, breasts, chest, stomach, armpit, lateral side, shoulder, arm, elbow, wrist, hand, finger, hip, buttocks, neck, torso, thigh, knee, calf, ankle, heel, foot, toe, head, chin, nose, jaw, or any other body location,) resulting from a surgery, procedure or injury, such as, for example, a breast surgery (e.g., cosmetic, reconstructive, mastectomy) or any chest area medical intervention (e.g., heart surgery, lung surgery, stomach surgery, armpit surgery, medical device implantation), whether for human males or human females, between ages 0 to 120. The integrated bandage system can be placed as a single all-encompassing bandage covering multiple adjacent or proximal locations, such as for example, both breasts (or a single breast), via a one-step application. The integrated bandage system can be non-compressive, partially compressive or contoured. The integrated bandage system can host gauze and adhesive being incorporated thereinto in order to effectively avoid, minimize, or reduce a need to separately apply those items to or in proximity to the wound area. The bandage system also may include drainage ports to allow blood and other liquids (e.g., lymph) to drain and be removed from the wound area. Resultantly, the integrated bandage system has at least one or any combination of (1) an ability to be self-applied (e.g., adhesive strip around front perimeter and not wrapping around wearer body), (2) an ability to be applied by others (e.g., adhesive strip around front perimeter (also sides and back), and wrapping around body (fully or partially)) or may include shoulder straps or connector hooks in the back, (3) expansion zones to accommodate swelling of breast tissue (e.g., located in various areas including areola reservoir portion), (4) expansion zones being adjustable with removable/adjustable pads, (5) anti-bacterial compositions included in the gauze, (6) anti-inflammation compositions included in the gauze, (7) various medicaments included in the gauze, (8) made out of or include all gauze, a combination of gauze and other more structured material, or a gauze insert, (9) the gauze can be insertable or may be replaceable, have different ways of adhering (flexible and malleable), (10) infection detection component incorporated into the device or coated onto the device, and (11) non-compressive or partially compressive. This disclosure is now described more fully with reference to a set of accompanying illustrative drawings, in which various example embodiments of this disclosure are shown. This disclosure can be embodied in many different forms and should not be construed as necessarily being limited to the example embodiments disclosed herein. Rather, the example embodiments are provided so that this disclosure is thorough and complete, and fully conveys various concepts of this disclosure to those skilled in a relevant art.

Features described with respect to certain example embodiments can be combined and sub-combined in and/or with various other example embodiments. Also, different aspects and/or elements of example embodiments, as disclosed herein, can be combined and sub-combined in a similar manner as well. Further, some example embodiments, whether individually and/or collectively, can be components of a larger system, wherein other procedures can take precedence over and/or otherwise modify their application. Additionally, a number of steps can be required before, after, and/or concurrently with example embodiments, as disclosed herein. Note that any and/or all methods and/or processes, at least as disclosed herein, can be at least partially performed via at least one entity in any manner.

Various terminology used herein can imply direct or indirect, full or partial, temporary or permanent, action or inaction. For example, when an element is referred to as being “on,” “connected” or “coupled” to another element, then the element can be directly on, connected or coupled to the other element and/or intervening elements can be present, including indirect and/or direct variants. In contrast, when an element is referred to as being “directly connected” or “directly coupled” to another element, there are no intervening elements present.

Although the terms first, second, etc. can be used herein to describe various elements, components, regions, layers and/or sections, these elements, components, regions, layers and/or sections should not necessarily be limited by such terms. These terms are used to distinguish one element, component, region, layer or section from another element, component, region, layer or section. Thus, a first element, component, region, layer, or section discussed below could be termed a second element, component, region, layer, or section without departing from various teachings of this disclosure.

Various terminology used herein is for describing particular example embodiments and is not intended to be necessarily limiting of this disclosure. As used herein, various singular forms “a,” “an” and “the” are intended to include various plural forms as well, unless a context clearly indicates otherwise. Various terms “comprises,” “includes” and/or “comprising,” “including” when used in this specification, specify a presence of stated features, integers, steps, operations, elements, and/or components, but do not preclude the presence and/or addition of one or more other features, integers, steps, operations, elements, components, and/or groups thereof.

As used herein, a term “or” is intended to mean an inclusive “or” rather than an exclusive “or.” That is, unless specified otherwise, or clear from context, “X employs A or B” is intended to mean any of a set of natural inclusive permutations. That is, if X employs A; X employs B; or X employs both A and B, then “X employs A or B” is satisfied under any of the foregoing instances.

Example embodiments of this disclosure are described herein with reference to illustrations of idealized embodiments (and intermediate structures) of this disclosure. As such, variations from various illustrated shapes as a result, for example, of manufacturing techniques and/or tolerances, are to be expected. Thus, various example embodiments of this disclosure should not be construed as necessarily limited to various particular shapes of regions illustrated herein, but are to include deviations in shapes that result, for example, from manufacturing.

Any and/or all elements, as disclosed herein, can be formed from a same, structurally continuous piece, such as being unitary, and/or be separately manufactured and/or connected, such as being an assembly and/or modules. Any and/or all elements, as disclosed herein, can be manufactured via any manufacturing processes, whether additive manufacturing, subtractive manufacturing, and/or any other types of manufacturing. For example, some manufacturing processes include three dimensional (3D) printing, laser cutting, computer numerical control routing, milling, pressing, stamping, vacuum forming, hydroforming, injection molding, lithography, and so forth.

Any and/or all elements, as disclosed herein, can be and/or include, whether partially and/or fully, a solid, including a metal, a mineral, an amorphous material, a ceramic, a glass ceramic, an organic solid, such as wood and/or a polymer, such as rubber, a composite material, a semiconductor, a nanomaterial, a biomaterial and/or any combinations thereof. Any and/or all elements, as disclosed herein, can be and/or include, whether partially and/or fully, a coating, including an informational coating, such as ink, an adhesive coating, a melt-adhesive coating, such as vacuum seal and/or heat seal, a release coating, such as tape liner, a low surface energy coating, an optical coating, such as for tint, color, hue, saturation, tone, shade, transparency, translucency, opaqueness, luminescence, reflection, phosphorescence, anti-reflection and/or holography, a photo-sensitive coating, an electronic and/or thermal property coating, such as for passivity, insulation, resistance or conduction, a magnetic coating, a water-resistant and/or waterproof coating, a scent coating and/or any combinations thereof. Any and/or all elements, as disclosed herein, can be rigid, flexible, and/or any other combinations thereof. Any and/or all elements, as disclosed herein, can be identical and/or different from each other in material, shape, size, color and/or any measurable dimension, such as length, width, height, depth, area, orientation, perimeter, volume, breadth, density, temperature, resistance, and so forth.

Unless otherwise defined, all terms (including technical and scientific terms) used herein have the same meaning as commonly understood by one of ordinary skill in an art to which this disclosure belongs. Various terms, such as those defined in commonly used dictionaries, should be interpreted as having a meaning that is consistent with a meaning in a context of a relevant art and should not be interpreted in an idealized and/or overly formal sense unless expressly so defined herein.

Furthermore, relative terms such as “below,” “lower,” “above,” and “upper” can be used herein to describe one element's relationship to another element as illustrated in the set of accompanying illustrative drawings. Such relative terms are intended to encompass different orientations of illustrated technologies in addition to an orientation depicted in the set of accompanying illustrative drawings. For example, if a device in the set of accompanying illustrative drawings were turned over, then various elements described as being on a “lower” side of other elements would then be oriented on “upper” sides of other elements. Similarly, if a device in one of illustrative figures were turned over, then various elements described as “below” or “beneath” other elements would then be oriented “above” other elements. Therefore, various example terms “below” and “lower” can encompass both an orientation of above and below.

As used herein, a term “about” and/or “substantially” refers to a +/−10% variation from a nominal value/term. Such variation is always included in any given value/term provided herein, whether or not such variation is specifically referred thereto.

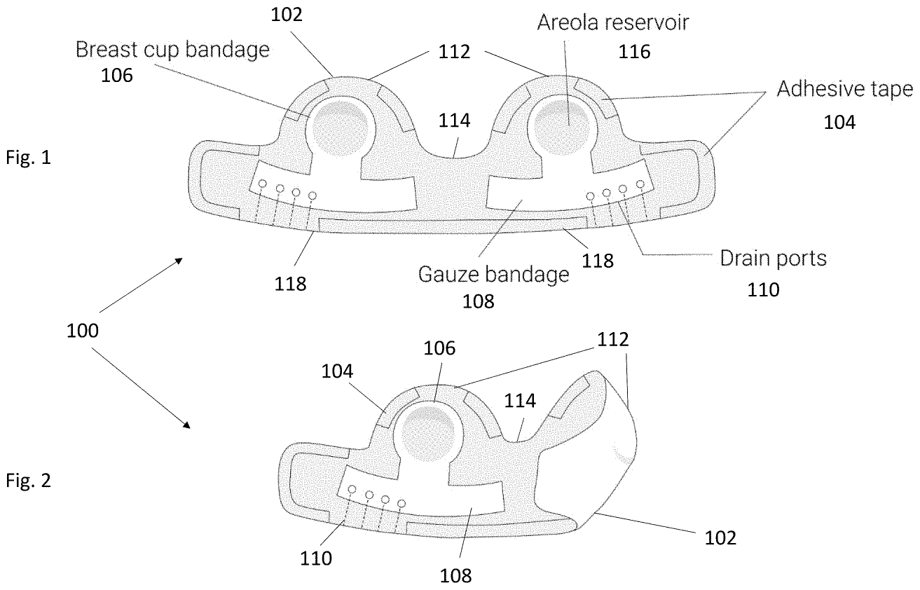

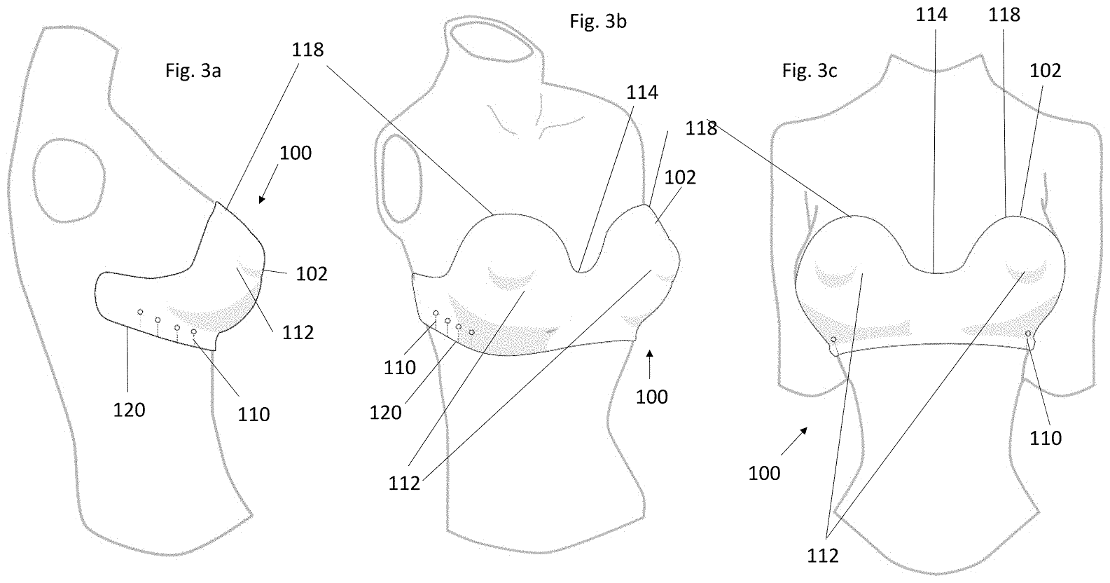

FIG. 1 illustrates a rear view of an embodiment of a bandage according to this disclosure. FIG. 2 illustrates a lateral view of an embodiment of a bandage according to this disclosure. FIGS. 3a-3c illustrate various views of an embodiment of a bandage on a torso of a mannequin or a human according to this disclosure. In particular, as shown in FIG. 1, a breast bandage 100 includes a body 102 having a plurality of sides 118 (left and right) and a central portion 114 spanning therebetween. The central portion 114 can be assembled with the sides 118 (e.g., stitched, weaved, hook-and-looped, zippered, buttoned, fastened, mated, interlocked, adhered, magnetized) or be unitary with the sides 118 (e.g., single piece of same material). The body 102 is structured such that the sides 118 are identical to each other in shape and size and therefore the sides 118 can fold onto themselves about the central portion 114 (e.g., living hinge style) or be symmetrical along a sagittal plane of a wearer of the breast bandage 100, as shown in FIG. 3c. However, note that the sides 118 can avoid being identical to each other in shape or size (e.g., vary in shape or size) and therefore avoid being symmetrical along the sagittal plane of the wearer of the breast bandage 100. The body 102, inclusive of the sides 118 and the central portion 114, can include an elastic material, elastomeric material, resilient material, shape memory material, flexible material, rigid material, fabric, net, yarn, mesh, textile, plant fiber, hand or machine spun fiber, cloth, gauze, silk, cotton, sponge, absorbent material, plastic, rubber, metal, film, Telfa, polyblend, zinc oxide, calamine, synthetic fiber, wire, or another material.

As shown in FIGS. 1 and 2, each of the sides 118 has a cup portion 112 hosting an areola reservoir portion 116 and a gauze bandage portion 108 extending about the areola reservoir portion 116. Although the cup portion 112 is semi-circular, the cup portion 112 can be shaped differently (e.g., open-shaped, closed-shape, D-shape, P-shape, O-shape, B-shape, square shape, triangle shape, oval shape, circular shape, rectangle shape, pentagon shape, octagon shape, J-shape, L-shape, C-shape, U-shape, F-shape, Y-shape, S-shape, Z-shape), whether symmetrical or asymmetrical. The cup portion 112 is sized and shaped to host a breast of the wearer of the breast bandage 100 (e.g., inwardly dimpled, inwardly conical). The cup portion 112 can include an elastic material, elastomeric material, resilient material, shape memory material, flexible material, rigid material, fabric, net, yarn, mesh, textile, plant fiber, hand or machine spun fiber, cloth, gauze, silk, cotton, sponge, absorbent material, plastic, rubber, metal, film, Telfa, polyblend, zinc oxide, calamine, synthetic fiber, wire, or another material. The cup portion 112 can host an expansion zone to accommodate swelling of a breast tissue when the wearer wears the breast bandage 100. The expansion zone can be adjustable with a pad.

The areola reservoir portion 116 is sized and shaped (e.g., inwardly dimpled, inwardly conical) to host an areola of the wearer of the breast bandage 100. The areola reservoir portion 116 can include an elastic material, elastomeric material, resilient material, shape memory material, flexible material, rigid material, fabric, net, yarn, mesh, textile, plant fiber, hand or machine spun fiber, cloth, gauze, silk, cotton, sponge, absorbent material, plastic, rubber, metal, film, Telfa, polyblend, zinc oxide, calamine, synthetic fiber, wire, or another material. The areola reservoir portion 116 can be assembled with that respective cup portion 112 (e.g., stitched, weaved, hook-and-looped, zippered, buttoned, fastened, mated, interlocked, adhered, bonded, magnetized) or be unitary with that respective cup portion 112 (e.g., single piece of same material). The areola reservoir portion 116 can host an expansion zone to accommodate swelling of a breast tissue when the wearer wears the breast bandage 100. The expansion zone can be adjustable with a pad.

As shown in FIG. 2, the gauze bandage portion 108 has a breast cup bandage portion 106 that extends about the areola reservoir portion 116 in a lollipop-cut manner (e.g., T-shape with O-shape attached thereto). For example, the breast cup bandage portion 106 can extend in the lollipop-cut manner when the gauze bandage portion 108 includes a ring portion, a stem portion, and a base portion, where the ring portion extends about the areola reservoir portion 116 and where the stem portion and the base portion form a T-shape. However, note that this form of extension can vary and the breast cup bandage portion 106 can extend about the areola reservoir portion 116 in an open-shaped manner (e.g., J-shape, L-shape, C-shape, U-shape, F-shape, Y-shape, S-shape, Z-shape) or a closed-shape manner (e.g., D-shape, P-shape, O-shape, B-shape, square shape, triangle shape, oval shape, circular shape, rectangle shape, pentagon shape, octagon shape), whether symmetrical or asymmetrical. The gauze bandage portion 108, inclusive of the breast cup bandage portion 106, can be assembled with that respective side 118 (e.g., stitched, weaved, hook-and-looped, zippered, buttoned, fastened, mated, interlocked, adhered, bonded, magnetized) or be unitary with that respective side 118 (e.g., single piece of same material). Note the gauze bandage portion 108, inclusive of the breast cup bandage portion 106, can extend into the central portion or past the central portion 114 into an opposing side 118 of the body 102. Also, note that the gauze bandage portions 108, inclusive of the breast cup bandage portions 106, for both of the cup portions 112, can be a single gauze bandage portion 108 such that the central portion 114 (e.g., valley) is lacking or that the single gauze bandage portion 108 extends into central portion 114 and past the central portion 114 into an opposing side 118 of the body 102. The gauze bandage portion 108, inclusive of the breast cup bandage portion 106, can host an expansion zone to accommodate swelling of a breast tissue when the wearer wears the breast bandage 100. The expansion zone can be adjustable with a pad.

The gauze bandage portion 108, inclusive of the breast cup bandage portion 106, can host an anti-bacterial composition (e.g., antibiotic), an anti-inflammation composition (e.g., antileukotrines, ImSAID, NSAID), a medicament (e.g., analgesic, breast cancer drug), an anti-viral composition (e.g., monoclonal antibody, amantadine, abacavir, acyclovir), an anti-fungal composition (e.g., polyene, azole), or an anti-parasitic composition (e.g., antiprotozoals, antinematodes). The gauze bandage portion 108, inclusive of the breast cup bandage portion 106, can be removable or detachable from the breast bandage 100.

Each of the sides 118 has a plurality of adhesive tape portions 104 peripherally extending therealong in a distributed and segmented manner. For example, prior to the breast bandage 100 being worn, at least one of the adhesive tape portions 104, which can be a strip thereof, can be covered with a corresponding cover tape, which can be peeled off that respective adhesive tape portion 100 to reveal that respective adhesive tape portion 100. The adhesive tape portions 104 extend external to the areola reservoir portions 116. For example, the adhesive tape portions 104 can extend about the areola reservoir portion 116 in an open-shaped manner (e.g., J-shape, L-shape, C-shape, U-shape, F-shape, Y-shape, S-shape, Z-shape) or a closed-shape manner (e.g., D-shape, P-shape, O-shape, B-shape, square shape, triangle shape, oval shape, circular shape, rectangle shape, pentagon shape, octagon shape), either of which can be symmetrical or asymmetrical. However, note that the adhesive tape strip portions 104 can be a single adhesive strip portion 104 peripherally extending along that respective side 118, which can be along a perimeter thereof, inclusive of an entire perimeter thereof. The adhesive tape portions 104 can be assembled with that respective side 118 (e.g., stitched, weaved, hook-and-looped, zippered, buttoned, fastened, mated, interlocked, adhered, bonded, magnetized) or be unitary with that respective side 118 (e.g., single piece of same material). For example, since the breast bandage 100 has an inner side, as shown in FIGS. 1 and 2, that faces the wearer when the wearer wears the breast bandage 100 and the inner side has a perimeter, then the adhesive tape portions 104 can extend along the perimeter. Note that the cup portion 112 can host at least one of the adhesive tape portions 104.

Each of the sides 118 includes a plurality of drain ports 110 (e.g., conduits, tubes, channels, inlets, outlets). The drain ports 110 can include an elastic material, elastomeric material, resilient material, shape memory material, flexible material, rigid material, fabric, net, yarn, mesh, textile, plant fiber, hand or machine spun fiber, cloth, gauze, silk, cotton, sponge, absorbent material, plastic, rubber, metal, film, Telfa, polyblend, zinc oxide, calamine, synthetic fiber, wire, or another material. The drain ports 110 can be rectilinear or non-rectilinear (e.g., arcuate, sinusoidal). The drain ports 110 can be assembled with that respective side 118 (e.g., stitched, weaved, hook-and-looped, zippered, buttoned, fastened, mated, interlocked, adhered, bonded, magnetized) or be unitary with that respective side 118 (e.g., single piece of same material). As shown in FIGS. 1 and 2, for each of the sides 118, the drain ports 110 can start from the gauze bandage portion 108, below the areola reservoir portion 116, and extend between the adhesive tape portions 104 toward an edge portion of that respective side 118. However, note that this form of extension can vary and the drain ports 110 can extend over or under the adhesive tape portions 104 or start at the areola reservoir portion 116 or start external to the gauze bandage portion 108 or start from at least one of the adhesive tape portions 104. In one or more embodiments, there is a single drain port 110 (e.g., conduit, tube, channel, inlet, outlet).

As better shown in FIGS. 3a-3c, each of the sides 118 includes a lateral side portion 120, which extends lateral to the cup portion 112 and along a lateral side of the wearer of the breast bandage 100 underneath or below an armpit of the wearer. However, note that the lateral side portion 120 can extend along the lateral side while not being underneath the armpit. The lateral side portion 120 hosts the drain ports 110. The lateral side portion 120 can include an elastic material, elastomeric material, resilient material, shape memory material, flexible material, rigid material, fabric, net, yarn, mesh, textile, plant fiber, hand or machine spun fiber, cloth, gauze, silk, cotton, sponge, absorbent material, plastic, rubber, metal, film, Telfa, polyblend, zinc oxide, calamine, synthetic fiber, wire, or another material. The lateral portion 120 can host the gauze bandage portion 108 or at least one of the adhesive tape portions 104. The adhesive tape portions 104 can be assembled with that respective lateral side portion 120 (e.g., stitched, weaved, hook-and-looped, zippered, buttoned, fastened, mated, interlocked, adhered, bonded, magnetized) or be unitary with that respective lateral side portion 120 (e.g., single piece of same material). As such, the breast bandage 100, inclusive of the lateral side portion 120, can extend along the lateral side of the wearer underneath the armpit of the wearer when the wearer wears the breast bandage 100, where the lateral side can include the drain ports 110 extending away from the armpit when the wearer wears the breast bandage 100.

As shown in FIGS. 1-3c, the breast bandage 100 does not have a back side that faces a back (e.g., spine, rib cage) of the wearer of the breast bandage 100 when the wearer wears the breast bandage 100 such that the breast bandage 100 is backless. Likewise, as shown in FIGS. 1-3c, the breast bandage 100 does not have a plurality of shoulder straps that extend over a plurality of shoulders of the wearer of the breast bandage 100 when the wearer wears the breast bandage 100 such that the breast bandage is strapless. Note that the breast bandage 100 can also avoid wrapping around a torso of the wearer when the wearer wears the breast bandage 100 (e.g., two sides, three sides, four sides).

The breast bandage 100 can include a back side that faces a back (e.g., spine, rib cage) of the wearer of the breast bandage 100 when the wearer wears the breast bandage 100. For example, the back side can host the adhesive tape portion 104. Additionally, as shown in FIGS. 1-3c, the breast bandage 100 can wrap around a torso of the wearer when the wearer wears the breast bandage 100. For example, when the torso has four sides, then the breast bandage 100 can wrap around two, three, or four of the four sides of the torso. For example, when the breast bandage 100 wraps around the four sides of the torso, then the breast bandage 100 can include a first engagement interface (e.g., hook-and-loop-hooks, buttons, magnets, adhesive) and a second engagement interface (e.g., hook-and-loop-hooks, buttons, magnets, adhesive), where the first engagement interface engages (e.g., fastens, mates, interlocks) the second engagement interface at any of the four sides when the wearer wears the breast bandage 100.

In one or more embodiments, the breast bandage 100 is non-compressive when adhered to the torso. In one or more embodiments, the breast bandage 100 may be non-compressive to the entire breast on which the surgery was performed or that has the incision. In one or more embodiments, the breast bandage 100 may be non-compressive to the portion of the breast on which the surgery was performed or that has the incision. In one or more embodiments, the breast bandage 100 remains non-compressive when the breast or breast tissue undergoes post-surgical or any swelling or expansion. In one or more embodiments, the breast bandage 100 remains non-compressive during the entire period or a portion of the entire period when the breast or breast tissue undergoes post-surgical or any swelling or expansion.

The breast bandage 100 can host a shoulder strap (e.g., from side 118, cup portion 112) that extends over the shoulder of the wearer when the wearer wears the breast bandage 100, which can couple to the back side of the breast bandage 100 or to an article of clothing or garment or medical device (e.g., cast, vest, shirt, implantable, wearable) worn by the wearer of the breast bandage 100. Also, note that the breast bandage 100 can include a strap (e.g., lasso style) that extends about a neck of the wearer when the breast bandage 100 is worn. Moreover, the breast bandage portion 100, inclusive of the body 102, inclusive of the sides 118, include of the cup portions 112, the central portion 114, the gauze bandage portion 108, the breast cup bandage portion 106, the adhesive tape portion 104, or the drain ports 110, can host at least one of a bacteria sensor (e.g., litmus paper style) or a virus sensor (e.g., litmus paper style), which can be a coating.

The breast bandage 100 can include a lower portion that extends underneath a breast of the wearer (e.g., over rib cage, past rib cage, over stomach) when the wearer wears the breast bandage 100. As such, the lower portion can include the adhesive tape portion 104 or the gauze bandage portion 108.

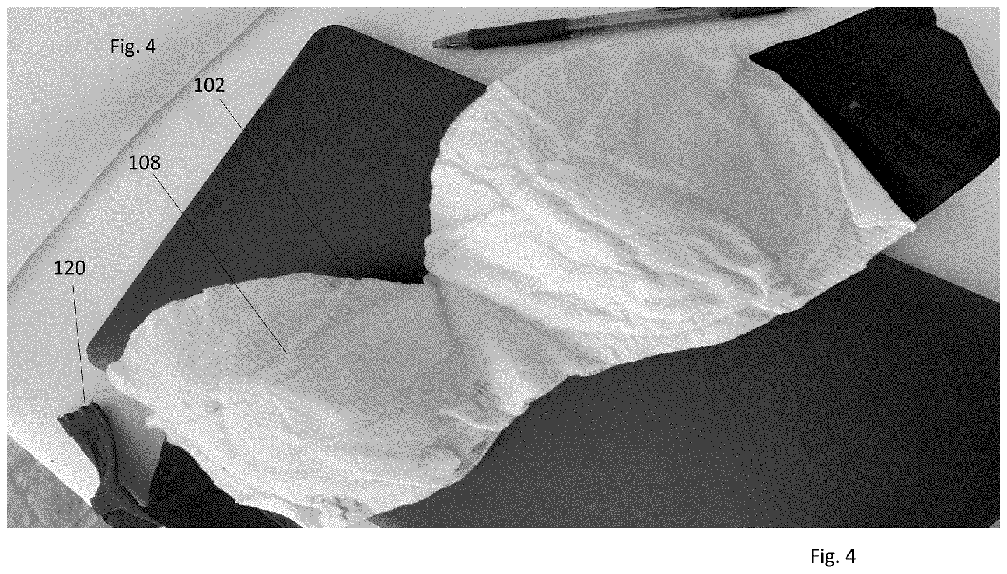

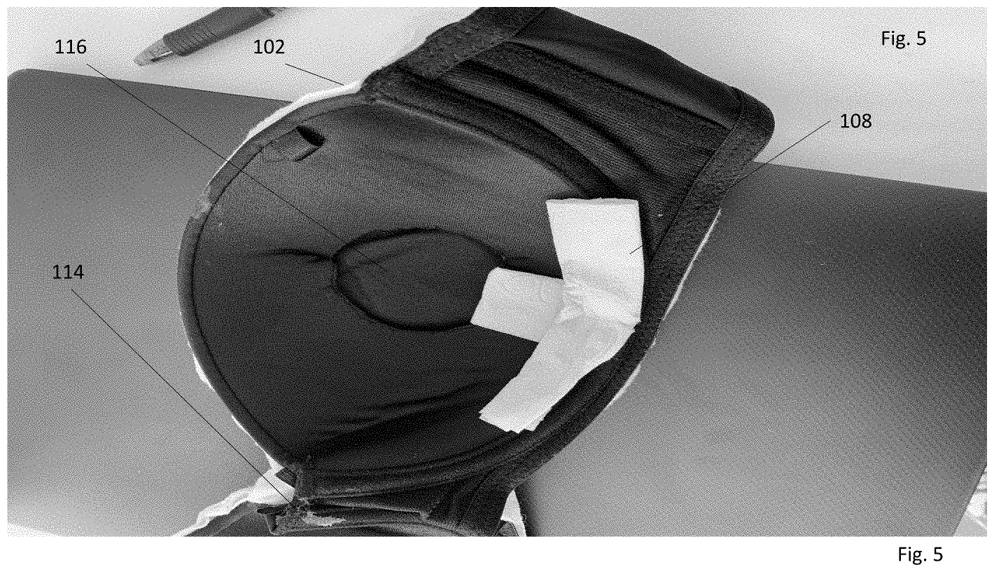

FIG. 4 illustrates a rear view of an embodiment of a bandage according to this disclosure. In particular, the body 102 hosts the gauze bandage portions 108 positioned over the cup portions 112 and the areola reservoir portions 116. Note that the body 102 has the lateral side portions 120, which can extend over the back portion of the wearer of the breast bandage 100.

FIG. 5 illustrates a rear view of an embodiment of a bandage according to this disclosure. In particular, the body 102 hosts the gauze bandage portion 108 positioned in the cup portion 112 and up to the areola reservoir portion 116. Note that the body 102 has the central portion 114. Further, note that the areola reservoir portion 116 allows or provide for a volume of space for swelling post the breast surgery, which can happen. Moreover, note that there is no gauze bandage portion 108 around an area of the breast bandage 100 corresponding to a breast area of where an incision (e.g., lollipop cut) of the breast surgery took place. This is to show at least some depth of the areola reservoir portion 116.

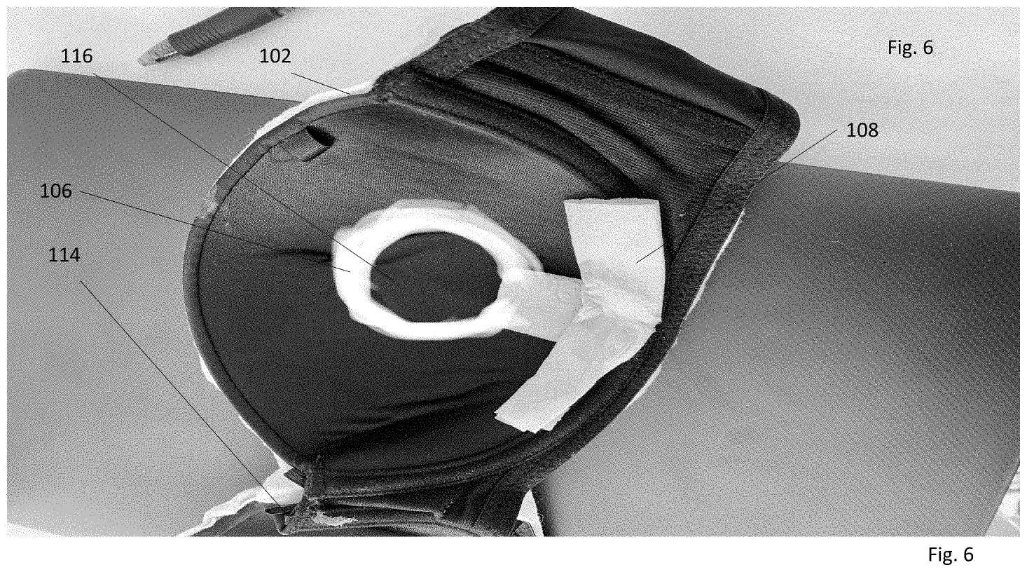

FIG. 6 illustrates a rear view of an embodiment of a bandage according to this disclosure. In particular, the body 102 hosts the gauze bandage portion 108 positioned in the cup portion 112 and up to the areola reservoir portion 116. Note that the body 102 has the central portion 114. Likewise, note that the breast cup bandage portion 106 (e.g., absorbable material) extends around the areola reservoir portion 116 (e.g., corresponding to breast area of lollipop incision) and is a part of at least some absorbable material (e.g., gauze bandage portion 108) included with an anchor shape and a vertical incision.

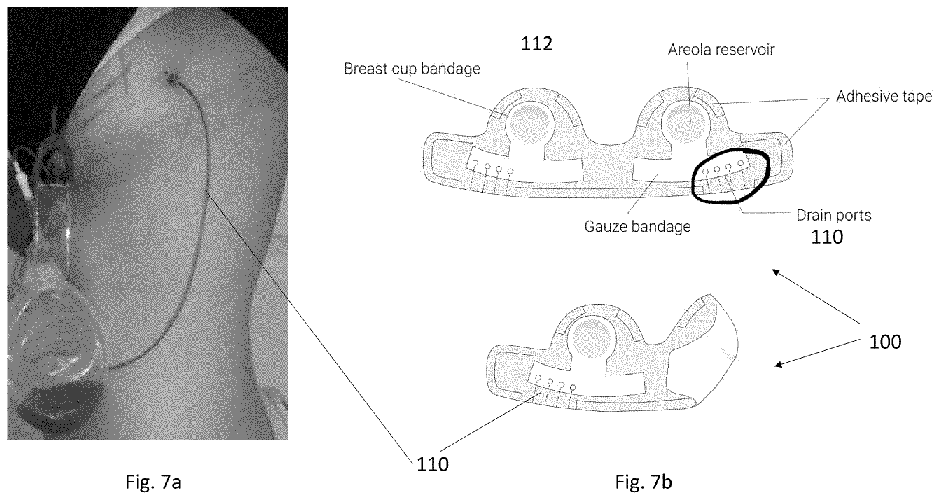

FIGS. 7a-7b illustrate an embodiment of usage of a drain port according to this disclosure. In particular, FIG. 7a shows how a drain tube laterally comes out of a skin of the wearer of the breast bandage 100 (from left side). The drain tube extends out laterally to a surgical incision (e.g., lollipop incision). Note that an exit side of the drain tube may vary and therefore the breast bandage 100 includes the drain ports 110 for at least some drainage to exit through, as shown in FIG. 7b. Note that the breast bandage 100 can be a complete one piece dressing with various features, as disclosed herein. Again, note that the breast bandage 100 can be non-circumferential or circumferential, with or without shoulder or neck straps.

The gauze bandage portions 108 may be adjusted to accommodate different locations of the incisions from the breast surgery. Such adjustments may be made by modifying the location of inserts or pads. Alternatively, such adjustments may be made by modifying the thickness or location of the gauze bandage portions 108.

If any disclosures are incorporated herein by reference and such disclosures conflict in part and/or in whole with this disclosure, then to an extent of a conflict, if any, and/or a broader disclosure, and/or broader definition of terms, this disclosure controls. If such disclosures conflict in part and/or in whole with one another, then to an extent of a conflict, if any, a later-dated disclosure controls.

The corresponding structures, materials, acts, and equivalents of all means or step plus function elements in the claims below are intended to include any structure, material, or act for performing the function in combination with other claimed elements as specifically claimed. The embodiments were chosen and described in order to best explain the principles of the disclosure and the practical application, and to enable others of ordinary skill in the art to understand the disclosure for various embodiments with various modifications as are suited to the particular use contemplated.

The diagrams depicted herein are illustrative. There can be many variations to the diagram or the steps (or operations) described therein without departing from the spirit of the disclosure. For instance, the steps can be performed in a differing order or steps can be added, deleted or modified. All of these variations are considered a part of the disclosure. It will be understood that those skilled in the art, both now and in the future, can make various improvements and enhancements which fall within the scope of the claims which follow.

In one or more embodiments, the bandage is shaped such that it can be used for dressing a chest wound, a stomach wound, an armpit wound, a lateral side wound, a shoulder wound, an arm wound, an elbow wound, a wrist wound, a hand wound, a finger wound, a hip wound, a buttocks wound, a neck wound, a torso wound, a thigh wound, a knee wound, a calf wound, an ankle wound, a heel wound, a foot wound, a toe wound, a head wound, a chin wound, a nose wound, a jaw wound, or to dress a wound in any other body location. Such a bandage includes some or all of the features of the breast bandages described above.

The description of this disclosure has been presented for purposes of illustration and description, but is not intended to be fully exhaustive and/or limited to the disclosure in the form disclosed. Many modifications and variations in techniques and structures will be apparent to those of ordinary skill in an art without departing from a scope and spirit of this disclosure as set forth in the claims that follow. Accordingly, such modifications and variations are contemplated as being a part of this disclosure. A scope of this disclosure is defined by various claims, which include known equivalents and unforeseeable equivalents at a time of filing of this disclosure.

What is claimed is:

1. A device comprising:
a breast bandage hosting a cup portion, a reservoir portion, a gauze portion, and an adhesive portion, wherein the cup portion hosts the reservoir portion, wherein the gauze portion extends about the reservoir portion, wherein the adhesive portion is external to the reservoir portion.

2. The device of claim 1, wherein the breast bandage includes a first side and a second side, wherein the first side includes the cup portion, the reservoir portion, the gauze portion, and the adhesive portion, wherein the first side and the second side are symmetrical along a sagittal plane of a wearer of the breast bandage when the wearer wears the breast bandage.

3. The device of claim 2, wherein the cup portion is a first cup portion, wherein the reservoir portion is a first reservoir portion, wherein the gauze portion is a first gauze portion, wherein the adhesive portion is a first adhesive portion, wherein the second side hosts a second cup portion, a second reservoir portion, a second gauze portion, and a second adhesive portion, wherein the second cup portion hosts the second reservoir portion, wherein the second gauze portion extends about the second reservoir portion, wherein the second adhesive portion is external to the second reservoir portion.

4. The device of claim 1, wherein the breast bandage includes a drain port.

5. The device of claim 1, wherein the breast bandage has an inner side that faces a wearer when the wearer wears the breast bandage, wherein the inner side has a perimeter, wherein the adhesive portion extends along the perimeter.

6. The device of claim 1, wherein the breast bandage does not have a back side that faces a back of a wearer of the breast bandage when the wearer wears the breast bandage such that the breast bandage is backless.

7. The device of claim 1, wherein the breast bandage does not have a plurality of straps that extend over a plurality of shoulders of a wearer of the breast bandage when the wearer wears the breast bandage such that the breast bandage is strapless.

8. The device of claim 1, wherein the breast bandage extends along a lateral side of a wearer below an armpit of the wearer when the wearer wears the breast bandage.

9. The device of claim 8, wherein the lateral side hosts the adhesive portion.

10. The device of claim 1, wherein the breast bandage does not wrap around a torso of a wearer when the wearer wears the breast bandage.

11. The device of claim 1, wherein the breast bandage includes a back side that faces a back of a wearer of the breast bandage when the wearer wears the breast bandage.

12. The device of claim 11, wherein the back side hosts the adhesive portion.

13. The device of claim 1, wherein the breast bandage wraps around a torso of a wearer when the wearer wears the breast bandage.

14. The device of claim 13, wherein the torso has four sides, wherein the breast bandage wraps around two of the four sides of the torso.

15. The device of claim 14, wherein the breast bandage wraps around three of the four sides of the torso.

16. The device of claim 15, wherein the breast bandage wraps around four of the four sides of the torso.

17. The device of claim 16, wherein the breast bandage has a first engagement interface and a second engagement interface, wherein the first engagement interface engages the second engagement interface at any of the four sides when the wearer wears the breast bandage.

18. The device of claim 1, wherein the breast bandage hosts a strap that extends over a shoulder of a wearer when the wearer wears the breast bandage.

19. The device of claim 1, wherein the breast bandage hosts an expansion zone to accommodate swelling of a breast tissue when a wearer wears the breast bandage.

20. The device of claim 19, wherein the expansion zone is adjustable with a pad.

21. The device of claim 1, wherein the gauze portion hosts an anti-bacterial composition.

22. The device of claim 1, wherein the gauze portion hosts an anti-inflammation composition.

23. The device of claim 1, wherein the gauze portion hosts a medicament.

24. The device of claim 1, wherein the gauze portion is removable from the breast bandage.

25. The device of claim 1, wherein the gauze portion is detachable from the breast bandage.

26. The device of claim 1, wherein the breast bandage includes a body, wherein the gauze portion adheres to the body.

27. The device of claim 26, wherein the gauze portion adheres to the body detachably.

28. The device of claim 1, wherein the breast bandage hosts at least one of a bacteria sensor or a virus sensor.

29. The device of claim 28, wherein the at least one of the bacteria sensor or the virus sensor is a coating.

30. The device of claim 1, wherein the gauze portion includes a ring portion, a stem portion, and a base portion, wherein the ring portion extends about the reservoir portion, wherein the stem portion and the base portion form a T-shape.

31. The device of claim 1, wherein the breast bandage extends along a lateral side of a wearer under an armpit of the wearer when the wearer wears the breast bandage, wherein the lateral side includes a plurality of drain ports extending away from the armpit when the wearer wears the breast bandage.

32. The device of claim 1, wherein the cup portion includes at least one of an elastic material or a resilient material.

33. The device of claim 1, wherein the cup portion includes the adhesive portion.

34. The device of claim 1, wherein the breast bandage includes a lateral portion that extends underneath an armpit of a wearer when the wearer wears the breast bandage.

35. The device of claim 34, wherein the gauze portion is a first gauze portion, wherein the lateral portion includes a second gauze portion.

36. The device of claim 1, wherein the breast bandage includes a lower portion that extends underneath a breast of a wearer when the wearer wears the breast bandage.

37. The device of claim 36, wherein the gauze portion is a first gauze portion, wherein the lower portion includes a second gauze portion.

38. The device of claim 1, wherein the breast bandage hosts a strap that extends over a neck of a wearer when the wearer wears the breast bandage.

39. The device of claim 1, wherein the gauze portion hosts an anti-viral composition.

40. The device of claim 1, wherein the gauze portion hosts an anti-fungal composition.

41. The device of claim 1, wherein the gauze portion hosts an anti-parasitic composition.
